SEQUENCE LISTING (12 sequences)

SEQ ID NO: 1 - DNA, 4107 bp, Artificial Sequence
  Ca9-coding sequence
atggacaagaagtacagcatcggcctggacatcggtaccaacagcgtgggctgggccgtgatcaccgacgagtacaaggtgcccagcaagaagttcaaggtgctgggcaacaccgaccgccacagcatcaagaagaacctgatcggcgccctgctgttcgacagcggcgagaccgccgaggccacccgcctgaagcgcaccgcccgccgccgctacacccgccgcaagaaccgcatctgctacctgcaggagatcttcagcaacgagatggccaaggtggacgacagcttcttccaccgcctggaggagagcttcctggtggaggaggacaagaagcacgagcgccaccccatcttcggcaacatcgtggacgaggtggcctaccacgagaagtaccccaccatctaccacctgcgcaagaagctggtggacagcaccgacaaggccgacctgcgcctgatctacctggccctggcccacatgatcaagttccgcggccacttcctgatcgagggcgacctgaaccccgacaacagcgacgtggacaagctgttcatccagctggtgcagacctacaaccagctgttcgaggagaaccccatcaacgccagcggcgtggacgccaaggccatcctgagcgcccgcctgagcaagagccgccgcctggagaacctgatcgcccagctgcccggcgagaagaagaacggcctgttcggcaacctgatcgccctgagcctgggcctgacccccaacttcaagagcaacttcgacctggccgaggacgccaagctgcagctgagcaaggacacctacgacgacgacctggacaacctgctggcccagatcggcgaccagtacgccgacctgttcctggccgccaagaacctgagcgacgccatcctgctgagcgacatcctgcgcgtgaacaccgagatcaccaaggcccccctgagcgccagcatgatcaagcgctacgacgagcaccaccaggacctgaccctgctgaaggccctggtgcgccagcagctgcccgagaagtacaaggagatcttcttcgaccagagcaagaacggctacgccggctacatcgacggcggcgccagccaggaggagttctacaagttcatcaagcccatcctggagaagatggacggcaccgaggagctgctggtgaagctgaaccgcgaggacctgctgcgcaagcagcgcaccttcgacaacggcagcatcccccaccagatccacctgggcgagctgcacgccatcctgcgccgccaggaggacttctaccccttcctgaaggacaaccgcgagaagatcgagaagatcctgaccttccgcatcccctactacgtgggccccctggcccgcggcaacagccgcttcgcctggatgacccgcaagagcgaggagaccatcaccccctggaacttcgaggaggtggtggacaagggcgccagcgcccagagcttcatcgagcgcatgaccaacttcgacaagaacctgcccaacgagaaggtgctgcccaagcacagcctgctgtacgagtacttcaccgtgtacaacgagctgaccaaggtgaagtacgtgaccgagggcatgcgcaagcccgccttcctgagcggcgagcagaagaaggccatcgtggacctgctgttcaagaccaaccgcaaggtgaccgtgaagcagctgaaggaggactacttcaagaagatcgagtgcttcgacagcgtggagatcagcggcgtggaggaccgcttcaacgccagcctgggcacctaccacgacctgctgaagatcatcaaggacaaggacttcctggacaacgaggagaacgaggacatcctggaggacatcgtgctgaccctgaccctgttcgaggaccgcgagatgatcgaggagcgcctgaagacctacgcccacctgttcgacgacaaggtgatgaagcagctgaagcgccgccgctacaccggctggggccgcctgagccgcaagcttatcaacggcatccgcgacaagcagagcggcaagaccatcctggacttcctgaagagcgacggcttcgccaaccgcaacttcatgcagctgatccacgacgacagcctgaccttcaaggaggacatccagaaggcccaggtgagcggccagggcgacagcctgcacgagcacatcgccaacctggccggcagccccgccatcaagaagggcatcctgcagaccgtgaaggtggtggacgagctggtgaaggtgatgggccgccacaagcccgagaacatcgtgatcgagatggcccgcgagaaccagaccacccagaagggccagaagaacagccgcgagcgcatgaagcgcatcgaggagggcatcaaggagctgggcagccagatcctgaaggagcaccccgtggagaacacccagctgcagaacgagaagctgtacctgtactacctgcagaacggccgcgacatgtacgtggaccaggagctggacatcaaccgcctgagcgactacgacgtggaccacatcgtgccccagagcttcctgaaggacgacagcatcgacaacaaggtgctgacccgcagcgacaagaaccgcggcaagagcgacaacgtgcccagcgaggaggtggtgaagaagatgaagaactactggcgccagctgctgaacgccaagctgatcacccagcgcaagttcgacaacctgaccaaggccgagcgcggcggcctgagcgagctggacaaggccggcttcatcaagcgccagctggtggagacccgccagatcaccaagcacgtggcccagatcctggacagccgcatgaacaccaagtacgacgagaacgacaagctgatccgcgaggtgaaggtgatcaccctgaagagcaagctggtgagcgacttccgcaaggacttccagttctacaaggtgcgcgagatcaacaactaccaccacgcccacgacgcctacctgaacgccgtggtgggcaccgccctgatcaagaagtaccccaagctggagagcgagttcgtgtacggcgactacaaggtgtacgacgtgcgcaagatgatcgccaagagcgagcaggagatcggcaaggccaccgccaagtacttcttctacagcaacatcatgaacttcttcaagaccgagatcaccctggccaacggcgagatccgcaagcgccccctgatcgagaccaacggcgagaccggcgagatcgtgtgggacaagggccgcgacttcgccaccgtgcgcaaggtgctgagcatgccccaggtgaacatcgtgaagaagaccgaggtgcagaccggcggcttcagcaaggagagcatcctgcccaagcgcaacagcgacaagctgatcgcccgcaagaaggactgggaccccaagaagtacggcggcttcgacagccccaccgtggcctacagcgtgctggtggtggccaaggtggagaagggcaagagcaagaagctgaagagcgtgaaggagctgctgggcatcaccatcatggagcgcagcagcttcgagaagaaccccatcgacttcctggaggccaagggctacaaggaggtgaagaaggacctgatcatcaagctgcccaagtacagcctgttcgagctggagaacggccgcaagcgcatgctggccagcgccggcgagctgcagaagggcaacgagctggccctgcccagcaagtacgtgaacttcctgtacctggccagccactacgagaagctgaagggcagccccgaggacaacgagcagaagcagctgttcgtggagcagcacaagcactacctggacgagatcatcgagcagatcagcgagttcagcaagcgcgtgatcctggccgacgccaacctggacaaggtgctgagcgcctacaacaagcaccgcgacaagcccatccgcgagcaggccgagaacatcatccacctgttcaccctgaccaacctgggcgcccccgccgccttcaagtacttcgacaccaccatcgaccgcaagcgctacaccagcaccaaggaggtgctggacgccaccctgatccaccagagcatcaccggtctgtacgagacccgcatcgacctgagccagctgggcggcgactaa

SEQ ID NO: 2 - protein, 1368 aa, Artificial Sequence [sequence omitted]

SEQ ID NO: 3 - DNA, 21 bp, Artificial Sequence
  Nuclear localization sequence or signal
cccaagaagaagaggaaggtg

SEQ ID NO: 4 - protein, 7 aa, Artificial Sequence
  Nuclear localization sequence or signal
PKKKRKV

SEQ ID NO: 5 - protein, 14 aa, Artificial Sequence
  LMWP(low molecular weight protamine)
VSRRRRRRGGRRRR

SEQ ID NO: 6 - protein, 11 aa, Artificial Sequence
  TAT
YGRKKRRQRRR

SEQ ID NO: 7 - protein, 16 aa, Artificial Sequence
  Penetratin
RQIKIWFQNRRMKWKK

SEQ ID NO: 8 - protein, 7 aa, Artificial Sequence
  polyarginine
RRRRRRR

SEQ ID NO: 9 - protein, 10 aa, Artificial Sequence
  polylysine
KKKKKKKKKK

SEQ ID NO: 10 - DNA, 59 bp, Artificial Sequence
  KRAS target site(EXON3)
tctcgacacagcaggtcaagaggagtacagtgcaatgagggaccagtacatgaggactg

SEQ ID NO: 11 - DNA, 20 bp, Artificial Sequence
  crRAN#1 targeting KRAS
tctcgacacagcaggtcaag

SEQ ID NO: 12 - DNA, 20 bp, Artificial Sequence
  crRNA#2 targeting KRAS
ggaccagtacatgaggactg

(19) United States
(12) Patent Application Publication
(10) Pub. No.: US 2019/0106688 A1
(43) Pub. Date: Apr. 11, 2019

(54) FUSION PROTEIN FOR CRISPR/CAS SYSTEM AND COMPLEX COMPRISING THE SAME AND USES THEREOF

(71) Applicant: KOREA INSTITUTE OF SCIENCE AND TECHNOLOGY, Seoul (KR)

(72) Inventors: Seung Ja OH, Seoul (KR); Mi Hue JANG, Seoul (KR); Seung Min KIM, Seoul (KR)

(73) Assignee: KOREA INSTITUTE OF SCIENCE AND TECHNOLOGY, Seoul (KR)

(21) Appl. No.: 16/156,864

(22) Filed: Oct. 10, 2018

(30) Foreign Application Priority Data
Oct. 11, 2017 (KR) 10-2017-0131637

(51) Int. Cl. C12N 9/22; C12N 15/11; A61K 38/46; A61K 45/06; A61K 47/64; A61K 47/55; A61K 47/54; A61P 35/00

(52) U.S. Cl. CPC C12N 9/22; C12N 15/11; A61K 38/465; A61K 45/06; A61K 47/64; C07K 2319/09; A61K 47/549; A61P 35/00; C12N 2800/80; C12N 2310/20; A61K 47/55

(57) ABSTRACT

Provided are a fusion protein used in a CRISPR/Cas system, a complex including the same, and uses thereof. The fusion protein may efficiently be used as an anticancer agent due to complexation with a guide RNA, remarkable intracellular delivery activity of the guide RNA in vivo or in vitro without any other cationic polymers or lipid carriers, and synergistic effects by co-administration with any other anticancer agent as well as anticancer activity by single treatment.

### CROSS-REFERENCE TO RELATED APPLICATION

This application claims the benefit of Korean Patent Application No. 10-2017-0131637, filed on Oct. 11, 2017, in the Korean Intellectual Property Office, the disclosure of which is incorporated herein in its entirety by reference.

### BACKGROUND

### 1. Field

One or more embodiments relate to a fusion protein for a CRISPR/Cas system, a complex including the same, and uses thereof.

### 2. Description of the Related Art

RNA-programmed Cas9 ribonucleoproteins (Cas9 RNPs) are adopted for mammalian gene editing with two small RNAs. A CRISPR RNA (crRNA) targets a desired genome sequence, and a small transactivating crRNA (tracrRNA) binds to the crRNA and Cas9. For genome editing, a chimeric single guide RNA (sgRNA) combines the crRNA and the tracrRNA. The use of the CRISPR-Cas9 system with purified Cas9 RNPs provides an innovative platform for highly efficient genome editing with fewer off-target cleavage than occur with plasmid- or viral-mediated delivery of Cas9 and sg RNAs, because the Cas9 RNP system directly introduces RNA or protein into the cells without requiring additional stages, such as transcription and translation. In general, Cas9 RNP-mediated delivery to target cells is carried out via lipid-mediated transfection or electroporation. According to a previous report, a cationic lipid-mediated delivery of Cas9 RNPs with sgRNA achieved up to a 20% genome modification in a mouse inner ear in vivo when it was complexed in 50% RNAiMAX or Lipofectamine 2000.1. Recently, a delivery of an engineered Cas9 with multiple SV40 nuclear localization sequences has been demonstrated for gene editing in a mouse brain in vivo. However, there are still challenges in Cas9 RNP-mediated gene editing in vivo. Particularly, since Cas9 RNPs have no intracellular delivery activity, their direct complexation and cellular internalization in vivo are necessary through conjugation with cationic polymers or lipid carriers, for which there remain several limitations with regard to the release of payloads into the cytoplasm, nuclear localization, and safety concerns.

As the most common cause of cancer-related death, non-small cell lung cancer (NSCLC) accounts for 80% of all lung cancers. NSCLC is a heterogeneous disease that may further be classified into three major subtypes, including adenocarcinoma, squamous cell carcinoma (SCC), and large cell carcinoma, which show distinct pathological characteristics. A number of genetic, epigenetic and signaling alterations underlie the development of lung cancers. Recently, the third generation EGFR inhibitor osimertinib has been approved for patients with metastasis resulting from a T790M mutation in EGFR.15 The development of the second generation ALK-targeting drugs is ongoing. Although KRAS is considered one of the most promising targets in NSCLC, there are still no FDA approved drugs targeting KRAS. Thus, there is need to develop an effective anti-KRAS therapy in NSCLC.

Thus, the present inventors have developed a fusion protein complexed with a guide RNA in a CRISPR/Cas system and delivered into cells without the aid of cationic polymers or lipid carriers and have identified that the fusion protein-guide RNA complex has anticancer activity alone or together with a targeted anticancer agent.

### SUMMARY

One or more embodiments include a fusion protein including a CRISPR-associated protein (Cas protein), a nuclear localization sequence (NLS), and/or a cationic cell penetrating peptide.

One or more embodiments include a polynucleotide encoding the fusion protein.

One or more embodiments include an expression vector including the polynucleotide.

One or more embodiments include a host cell transformed by the expression vector.

One or more embodiments include a composition for intracellular delivery of a guide RNA including the fusion protein.

One or more embodiments include a composition for formation of a complex with a guide RNA including the fusion protein.

One or more embodiments include a method of preparing a complex to deliver the guide RNA into a cell including brining the fusion protein into contact with the guide RNA.

One or more embodiments include a complex including the fusion protein and the guide RNA.

One or more embodiments include a composition for target gene-specific editing including the complex.

One or more embodiments include a method of editing a target gene, the method including administering the complex into an individual.

One or more embodiments include a pharmaceutical composition for prevention and treatment of cancer including the complex.

One or more embodiments include a method of preventing or treating cancer, the method including administering the complex into an individual.

One or more embodiments include a method of preparing the fusion protein or the complex.

One or more embodiments include a method of editing a target gene in a cell, the method including treating the cell with the complex.

Additional aspects will be set forth in part in the description which follows and, in part, will be apparent from the description, or may be learned by practice of the presented embodiments.

According to one or more embodiments, a fusion protein includes a CRISPR-associated protein (Cas protein), a nuclear localization sequence (NLS), and/or a cationic cell penetrating peptide.

The NLS and the cationic cell penetrating peptide may bind to a C-terminal of the Cas protein. Particularly, the NLS may bind to the C-terminal of the Cas protein, and the cationic cell penetrating peptide may bind to a C-terminal of the NLS or the C-terminal of the Cas protein.

In general, the term “CRISPR system” collectively refers to transcripts and other elements involved in expression of or directing the activity of CRISPR-associated (“Cas”) genes, including a Cas gene-encoding sequence, a trans-activating CRISPR (tracr) sequence (e.g., tracrRNA or active partial tracrRNA), a tracr-mate sequence (including “direct repeat” and a tracrRNA-processed partial direct repeat in the context of an endogenous CRISPR system), a guide sequence (also referred to as a “spacer” in the context of an endogenous CRISPR system), or other sequences and transcripts from a guide RNA or a CRISPR locus. In an embodiment, one or more elements of a CRISPR system is derived from type I, type II, or type III CRISPR system. According to the embodiment, at least one element of the CRISPR system is derived from a particular organism including an endogenous CRISPR system, for example, Streptococcus pyogenes. In general, the, CRISPR system is characterized by elements that promote formation of a CRISPR complex at a site of a target sequence (also referred to as a protospacer in the context of an endogenous CRISPR system). In the context of formation of a CRISPR complex, the term “target sequence” or “target gene” refers to a sequence to which a guide sequence is designed to have complementarity. In this regard, hybridization between a target sequence and a guide sequence promotes formation of the CRISPR complex. Although full complementarity is not necessarily required, there is sufficient complementarity to cause hybridization and promote formation of a CRISPR complex. A target sequence may include any polynucleotide, such as DNA or RNA polynucleotide. In some embodiments, a target sequence is located in the nucleus or cytoplasm of a cell. In some embodiments, the target sequence may be present in an organelle of a eukaryotic cell, such as, mitochondria or chloroplast.

When the Cas protein is complexed with two RNAs called CRISPR RNA (crRNA) and trans-activating crRNA (tracrRNA), activated endonuclease or nickase is formed. Examples of the Cas protein may include, but are not limited to, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes have been known. For example, an amino acid sequence of Streptococcus pyogenes Cas9 protein may be obtained from the SwissProt database with accession number Q99ZW2. In some embodiments, an unmodified CRISPR enzyme, e.g., Cas9, has DNA cleavage activity. In some embodiments, the CRISPR enzyme may be Cas9, e.g., Cas9 from Streptococcus pyogenes or Streptococcus pneumoniae. In some embodiments, the Cas protein is codon optimized for expression in eukaryotic cells. A coding sequence of the Cas9 may include a polynucleotide sequence having a homology of 80% or more with a polynucleotide sequence of SEQ ID NO: 1. In addition, the Cas9 may include an amino acid sequence of SEQ ID NO: 2 derived from Streptococcus pyogenes.

Throughout the specification, the term “nuclear localization sequence or signal (NLS)” refers to an amino acid sequence responsible for delivering a particular substance (e.g., protein) into a nucleus of a cell, mostly through nuclear pores (Kalderon D, et al., Cell 39:499509(1984); Dingwall C, et al., J Cell Biol. 107(3):8419(1988)). Although the NLS is not necessary for activation of the CRISPR complex in eukaryote, it is understood that these sequences promote the activity of the system, particularly to target nucleic acid molecules in the nucleus. The CRISPR enzyme may include at least one NLS having a sufficient strength to induce accumulation of a detectable amount of the CRISPR enzyme in the nucleus of a eukaryotic cell. The NLS may include a polynucleotide sequence of SEQ ID NO: 3, for example, a polynucleotide sequence having a homology of 80% or more. Particularly, the NLS may include an amino acid sequence of SEQ ID NO: 4.

The cationic cell penetrating peptide may include, for example, 70% or more, 80% or more, or 70% to 90% of at least one amino acid residue selected from arginine, lysine, and histidine over the entire amino acid sequence. In addition, the cell penetrating peptide may be an L-type or D-type peptide in view of stability in the body of living organisms. Particularly, the cationic cell penetrating peptide may have a length of 10 to 40 amino acid residues including at least one amino acid residue selected from arginine, lysine, and histidine. In an embodiment, the cell penetrating peptide may be a low molecular weight protamine (LMWP). Any cationic peptide other than the LMWP (SEQ ID NO: 5: VSRRRRRRGGRRRR), i.e., peptide mainly including arginine, lysine, or histidine may be used. For example, TAT (SEQ ID NO: 6: YGRKKRRQRRR), Penetratin (SEQ ID NO: 7: RQIKIWFQNRRMKWKK), polyarginine (SEQ ID NO: 8: RRRRRRR), polylysine (SEQ ID NO: 9: KKKKKKKKKK), protamine fragment, and Antennapedia (ANTP) may be used and any other peptide or peptide analogs other than the above-mentioned peptides may also be used as long as they may penetrate cell membranes.

In the fusion protein, the cationic cell penetrating peptide may be used to form a complex with the guide RNA. Also, the cationic cell penetrating peptide may be used to deliver the guide RNA into the cell.

Thus, according to one or more embodiments, a composition for intracellular delivery of a guide RNA includes a Cas protein, an NLS, and/or a cationic cell penetrating peptide into a cell. According to one or more embodiments, a composition for formation of a complex with a guide RNA includes a Cas protein; an NLS; and/or a cationic cell penetrating peptide.

According to one or more embodiments, an isolated nucleic acid molecule (e.g., polynucleotide) encodes the fusion protein.

According to one or more embodiments, an expression vector includes the nucleic acid molecule.

According to one or more embodiments, an expression vector includes the isolated nucleic acid molecule (e.g., polynucleotide) encoding the fusion protein, and a guide sequence (e.g., guide RNA).

According to one or more embodiments, a host cell transformed by the expression vector.

The term “polynucleotide” refers to a polymer of deoxyribonucleotide or ribonucleotide in either single- or double-stranded form. The polynucleotide includes RNA genome sequences, DNA (gDNA and cDNA) and RNA sequences transcribed therefrom, and analogues of natural polynucleotides unless otherwise stated.

The polynucleotide includes not only a nucleotide sequence encoding the amino acid sequence of the fusion protein but also a complementary sequence thereto. The complementary sequence includes not only a perfectly complementary sequence but also a substantially complementary sequence that may be hybridized with a sequence of a nucleotide encoding the amino acid sequence of the fusion protein under stringent conditions known in the art.

The term “vector” refers to a vehicle designed to express a target gene in a host cell. For example, the vector includes vectors such as a plasmid vector, a cosmid vector, a bacteriophage vector, an adenovirus vector, a retrovirus vector, and an adeno-associated virus vector. Vectors that may be used as the recombinant vector by manipulating plasmids such as pSC101, pGV1106, pACYC177, ColE1, pKT230, pME290, pBR322, pUC8/9, pUC6, pBD9, pHC79, pIJ61, pLAFR1, pHV14, pGEX series, and pET series, and pUC19, phage such as λgt4λB, λ-Charon, λΔz1, and M13, or virus such as SV40.

The polynucleotide sequence encoding the fusion protein in the recombinant vector is operatively linked to a promotor. The term “operatively linked” refers to functional linkage between a nucleotide expression control sequence such as a promotor and another nucleotide sequence. Thus, the control sequence regulates transcription and/or translation of the latter.

The recombinant vector may be an expression vector capable of stably producing the fusion protein in the host cell. The expression vector may be any known vector used to express foreign protein in plants, animals, or microorganisms. The recombinant vector may be constructed by using various methods well known in the art.

The recombinant vector may be constructed using a prokaryotic cell or a eukaryotic cell as a host. For example, when the vector according to the present embodiment is an expression vector and the prokaryotic cell is a host, the vector may generally include a strong promotor capable of promoting transcription (e.g., pLλpromoter, trp promoter, lac promoter, tac promoter, and T7 promoter), a ribosome binding site for initiation of translation, and a transcription/translation termination sequence. When the eukaryotic cell is used as a host, an origin of replication operating in the eukaryotic cell and included in the vector may include an f1 origin of replication, an SV40 origin of replication, a pMB1 origin of replication, an adeno origin of replication, an AAV origin of replication, and a BBV origin of replication, without being limited thereto. Also, a promotor derived from genome of mammalian cells (for example, metallothionein promoter) or a promotor derived from mammalian viruses (for example, adenovirus late promoter, vaccinia virus 7.5 K promoter, SV40 promoter, cytomegalovirus promoter, and tkpromoter of HSV) may be used, and the vector generally includes a polyadenylation sequence as the transcription termination sequence.

The cell, for example, the eukaryotic cell, may be a cell of yeast, fungus, protozoa, plant, higher plant, insect, and amphibian or mammalian such as CHO, HeLa, HEK293, and COS-1, for example, a cultured cell (in vitro), a graft cell, and a primarily cultured cell (in vitro and ex vivo), and an in vivo cell, and also a cell of mammals including humans which are commonly used in the art. In addition, the organism may be yeast, mold, protozoan, plant, higher plant, insect, amphibian, or mammal.

According to one or more embodiments, a complex (e.g., CRISPR complex) includes: a fusion protein including a Cas protein, an NLS; and/or a cationic cell penetrating peptide, and a guide RNA.

The terms “chimeric RNA”, “chimeric guide RNA”, “guide RNA”, “single guide RNA”, and “synthetic guide RNA” as used herein are used interchangeably and refer to a polynucleotide sequence including a guide sequence, a tracr sequence, and/or a tracr mate sequence. The term “guide sequence” as used herein refers to a sequence of about 20 base pairs (bp) in a guide RNA that specifies a target site and may be used interchangeably with the term “guide” or “spacer”. In addition, the term “tracr mate sequence” may be used interchangeably with “direct repeat(s)”. The guide RNA may be formed of two RNAs, that is, a CRISPR RNA (crRNA) and a transactivating crRNA (tracrRNA) or may be a single chain RNA (sgRNA) including portions of crRNA and tracrRNA and hybridizing with a target DNA.

In general, the guide sequence may be any polynucleotide sequence hybridizing with the target sequence with sufficient complementarity to the target polynucleotide sequence to induce CRISPR complex sequence-specific binding as the target sequence. In some embodiments, the degree of complementarity between the guide sequence and a target sequence corresponding thereto may be at least 50%, 60%, 75%, 80%, 85%, 90%, 95%, 97.5%, or 99% when optimally aligned using an appropriate alignment algorithm. The optimal alignment may be determined by using any suitable algorism for aligning sequences. Examples of the algorithm may include, but is not limited to, the Smith-Waterman algorithm, the Needleman-Wunsch algorithm, algorithms based on the Burrows-Wheeler Transform (e.g., Burrows Wheeler Aligner), ClustalW, Clustal X, BLAT, Novoalign (Novocraft Technologies), ELAND (Illumina, San Diego, Calif.), SOAP (available at soap.genomics.org.cn), and Maq (available at maq.sourceforge.net). In some embodiments, a guide sequence is about 5, 10, 11, 12, 13, 14, 15, 16, 17, 18, 19, 20, 21, 22, 23, 24, 25, 26, 27, 28, 29, 30, 35, 40, 45, 50, or 75 or more nucleotides in length. In some embodiments, the guide sequence is about 75, 50, 45, 40, 35, 30, 25, 20, 15, or 12 or less nucleotides in length. The ability of the guide sequence inducing the CRISPR complex sequence-specific binding to the target sequence may be evaluated by any suitable assay. For example, an element of the CRISPR system to form the CRISPR complex including the guide sequence to be tested may be provided to a host cell including a target sequence corresponding thereto as in evaluation of prior cleavage in the target sequence after transfection into a vector encoding the element of the CRISPR system by Surveyor assay as described herein. Similarly, cleavage of a target polynucleotide sequence may be evaluated in vitro by providing the target sequence, elements of a CRISPR complex including, a guide sequence to be tested, and a control guide sequence different from the tested guide sequence, and comparing binding or cleavage rates at the target sequence between the test and control guide sequence reactions. Other assays are possible and will occur to those skilled in the art.

The guide sequence may be selected to target any target sequence. In some embodiments, the target sequence is a sequence within a genome of a cell. For example, the target sequence includes those unique in a target genome. For example, for Streptococcus pyogenes Cas9, a unique target sequence in a genome may include a Cas9 target site of the form MMMMMMMMNNNNNNNNNNNNXGG, where NNNNNNNNNNNNXGG (N is A, G, T, or C; and X may be anything) has a single occurrence in the genome. A unique target sequence in a genome may include a Streptococcus pyogenes Cas9 target site of the form MMMMMMMMMNNNNNNNNNNNXGG, where NNNNNNNNNNNXGG (N is A, G, T, or C; and X may be anything) has a single occurrence in the genome. For Streptococcus thermophilus CRISPR1 Cas9, a unique target sequence in a genome may include a Cas9 target site of the form MMMMMMMMNNNNNNNNNNNNXXAGAAW, where NNNNNNNNNNNNXXAGAAW (N is A, G, T, or C; X may be anything; and W is A or T) has a single occurrence in the genome. A unique target sequence in a genome may include a Streptococcus thermophilus CRISPR1 Cas9 target site of the form MMMMMMMMMNNNNNNNNNNNXXAGAAW, where NNNNNNNNNNNXXAGAAW (N is A, G, T, or C; X may be anything; and W is A or T) has a single occurrence in the genome. For Streptococcus pyogenes Cas9, a unique target sequence in a genome may include a Car9 target site of the form MMMMMMMMNNNNNNNNNNNNXGGXG, where NNNNNNNNNNNNXGGXG (N is A, G, T, or C; and X may be anything) has a single occurrence in the genome. A unique target sequence in a genome may include a Streptococcus pyogenes Cas9 target site of the form MMMMMMMMMNNNNNNNNNNNXGGXG, where NNNNNNNNNNNXGGXG (N is A, G, T, or C; and X may be anything) has a single occurrence in the genome. In these sequences, “M” may be A, G, T, or C.

In the complex, crRNA may be linked to tracrRNA.

According to an embodiment, the complex may target EXON3 of KRAS, for example, a sequence of SEQ ID NO: 10. That is, the target sequence (target gene) may be a sequence of SEQ ID NO: 10 of KRAS. In addition, those of ordinary skill in the art may design a guide sequence targeting any target sequence as described above to obtain desired effects. For example, the guide sequence targeting the sequence of SEQ ID NO: 10 of KRAS may include 11 or 12 polynucleotides.

According to an embodiment, the cationic cell penetrating peptide enables formation of a complex between the fusion protein and the guide RNA via electrostatic interaction with the guide RNA without being limited by any particular theory. Thus, the complex may be self-assembled by the fusion protein to form a complex with the guide RNA.

According to one or more embodiments, a composition for target gene-specific editing includes the complex.

According to one or more embodiments, a pharmaceutical composition for prevention or treatment of cancer includes the complex.

According to one or more embodiments, a method of editing a target gene includes administering the complex to an individual.

According to one or more embodiments, a method of preventing or treating cancer includes administering the complex to an individual.

The terms “subject”, “individual”, and “patient” are used interchangeably herein to refer to a vertebrate, preferably, a mammal, more preferably. Mammals include, but are not limited to, murines, simians, humans, farm animals, sport animals, and pets. Tissues, cells, and their progeny of a biological entity obtained in vivo or incubated in vitro are also encompassed.

The term “therapeutic agent” or “pharmaceutical composition” refers to a molecule or compound that confers some beneficial effects upon administration to a subject. The beneficial effects include: enablement of diagnostic determinations; amelioration of a disease, symptom, disorder, or pathological condition; reducing or preventing the onset of a disease, symptom, disorder, or condition; and counteracting a disease, symptom, disorder, or pathological condition.

As used herein, the term “treatment” or “treating”, or “palliating” or “ameliorating” may be used interchangeably. These terms refer to methods of obtaining beneficial or desired results including, but not limited to, a therapeutic benefit and/or a prophylactic benefit. The therapeutic benefit indicates any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary according to one or more of a subject and disease condition being treated, a weight and age of the subject, a severity of the disease condition, and an administration method which may be easily determined by one of ordinary skill in the art. Also, the term may be applied to a dose that will provide an image for detection by any one of the imaging methods described herein. A particular dose may vary according to one or more of a particular agent chosen, a dosing regimen to be followed, whether it is administered in combination with other compounds, timing of administration, the tissue to be imaged, and the physical delivery system in which it is carried.

The cancer may be lung cancer, pancreatic cancer, gastric cancer, liver cancer, colon cancer, brain cancer, breast cancer, thyroid cancer, bladder cancer, esophageal cancer, or uterine cancer. Particularly, the cancer may be non-small-cell lung cancer. More particularly, the cancer may be KRAS-mutant cancer.

The composition may further include an anticancer agent, for example, a targeted anticancer agent. The anticancer agent may be an MEK inhibitor. The MEK inhibitor may refer to an anticancer agent that affects a mitogen-activated protein kinase (MAPK) pathway. The targeted anticancer agent may refer to an anticancer agent that selectively kills only cancer cells by blocking signals involved in the growth and development of cancer targeting specific proteins or specific gene changes that occur in particular cancer cells. The target of the targeted anticancer agent may be, for example, MEK or KRAS.

According to an embodiment, the complex has a synergistic effect when co-administered with the targeted anticancer agent.

According to one or more embodiments, a method of preparing the fusion protein or the complex is provided.

The method of preparing the fusion protein is as described above.

The method of preparing the complex may be performed by mixing the fusion protein with the guide RNA and culturing the mixture.

Particularly, the method may include: preparing an expression vector into which a polynucleotide encoding a fusion protein including a Cas protein, an NLS, and/or a cationic cell penetrating peptide is inserted; purifying the fusion protein from a host cell transformed by the expression vector; and culturing a mixture of the purified fusion protein and a guide RNA.

According to one or more embodiments, a method of editing a target gene in a cell including treating the cell with the complex.

### BRIEF DESCRIPTION OF THE DRAWINGS

The patent or application file contains at least one color drawing. Copies of this patent or patent application publication with color drawings will be provided by the USPTO upon request and payment of the necessary fee. These and/or other aspects will become apparent and more readily appreciated from the following description of the embodiments, taken in conjunction with the accompanying drawings in which:

FIG. 1A is a schematic diagram illustrating a vector to prepare triplexed Cas 9 RNPs according to an embodiment;

FIG. 1B is a diagram illustrating a structure of triplexed Cas 9 RNPs according to an embodiment, where LMWP is shown in red and fused to the C-terminal of Cas9 shown as a grey molecular surface;

FIG. 1C is a diagram illustrating electrostatic surface potential of Cas9 with dual RNAs and target DNA;

FIG. 2A is a diagram illustrating electrostatically induced complex formation of Cas9-LMWP fusion protein and dual RNA identified by a gel mobility shift assay;

FIG. 2B is a graph illustrating zeta potentials (red lines) and size distributions (blue lines) of resulting complexes respectively measured with a Zetasizer and DLS;

FIG. 2C shows scanning electron microscopic (SEM) images illustrating the morphologies, size distributions, and size homogeneity of triplexed Cas9 RNPs at two given ratios (1:1 or 1:5);

FIG. 2D shows transmission electron microscopic (TEM) images illustrating the morphologies, size distributions, and size homogeneity of triplexed Cas9 RNPs at two given ratios (1:1 or 1:5);

FIG. 2E shows atomic force microscopic (AFM) images illustrating the morphologies, size distributions, and size homogeneity of triplexed Cas9 RNPs at two given ratios (1:1 or 1:5);

FIG. 3 is a diagram illustrating immunogenicity of triplexed Cas9 RNP according to an embodiment, where *P<0.05, ** P<0.01, *** P<0.001 by a one-way ANOVA followed with Tukey's multiple comparison test;

FIG. 4A is a diagram illustrating frequencies of indel mutations in A549 cells treated with Cas9-LMWP and crRNA #1 or crRNA #2;

FIG. 4B is a diagram illustrating effects of triplexed Cas9 RNPs (72 pmol) on suppressing expression of KRAS identified by immunoblotting;

FIG. 4C illustrates confocal microscopic images indicating suppression of KRAS expression after direct delivery of triplexed Cas9 RNPs (72 pmol) (left panel) and a graph obtained by quantifying the results (right panel);

FIG. 4D illustrates cellular internalization of triplexed Cas9 RNPs (72 pmol), where green and red colors indicate locations of Cas9-LMWP from triplexed Cas9 RNPs and nuclei, respectively;

FIG. 4E is a graph illustrating viability of lung cancer cells according to various doses of triplexed Cas9 RNPs;

FIG. 4F illustrates FACS analysis results indicating apoptosis induced by triplexed Cas9 RNPs (72 pmol);

FIG. 4G illustrates confocal microscopic images indicating apoptosis induced by triplexed Cas9 RNPs (72 pmol) (left panel) and a graph obtained by quantifying the results (right panel);

FIG. 4H is a diagram illustrating that triplexed Cas9 RNPs (13.5 pmol) significantly suppresses cell migration, where *P<0.05, ** P<0.01, *** P<0.001 by a one-way ANOVA followed with Tukey's multiple comparison test;

FIG. 5A is a diagram illustrating a scheme of administering triplexed Cas9 RNPs and LF2000/Cas9 RNPs into A549 xenograft tumor models for comparison of capabilities of delivering substances for gene editing in vivo;

FIG. 5B illustrates confocal microscopic images showing results of administration of triplexed Cas9 RNPs and LF2000/Cas9 RNPs (upper panel) and graphs obtained by immunofluorescence staining analysis (lower panel). Bars indicate 50 μm, and inhibition of KRAS, p-ERK, and p-AKT was quantified using imageJ program (N=3), where *P <0.05, ** P<0.01, *** P<0.001 by a one-way ANOVA followed with Tukey's multiple comparison test;

FIG. 6A illustrates a scheme of administering triplexed Cas9 RNPs (0.4 mg/kg) and AZD6244 (25 mg/kg) by either single treatment or co-treatment (left panel) and photographs of tumors isolated from mice at 28 days after treatment;

FIG. 6B is a graph illustrating sizes of tumors after single treatment or co-treatment of the triplexed Cas9 RNPs (0.4 mg/kg) and AZD6244 (25 mg/kg), where *P <0.05, ** P<0.01, *** P<0.001 by a one-way ANOVA followed with Tukey's multiple comparison test;

FIG. 6C is a graph illustrating tumor growth inhibition (% TGI) for evaluation of antitumor efficiency, where *P<0.05, ** P<0.01, *** P<0.001 by a one-way ANOVA followed with Tukey's multiple comparison test;

FIG. 6D illustrates immunoblot analysis of EGFR downstream signaling pathways in A549 cells and A549 xenografts;

FIG. 6E illustrates tumor cells stained by H&E staining, TUNEL staining, and immunolabelling of p-ERK, KRAS, and p-AKT after single treatment or co-treatment of the triplexed Cas9 RNPs (0.4 mg/kg) and AZD6244 (25 mg/kg), where bars indicate 50 μm; and

FIG. 7 is a diagram illustrating triplexed Cas9 RNPs-mediated gene editing for cancer therapeutics according to an embodiment.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description. As used herein, the term “and/or” includes any and all combinations of one or more of the associated listed items. Expressions such as “at least one of,” when preceding a list of elements, modify the entire list of elements and do not modify the individual elements of the list.

### REFERENCE EXAMPLE

IFN-α and TNF-α Assay

Normal human peripheral blood mononuclear cells (PBMCs) were isolated from healthy donors (Innovative research) by standard Ficoll-Paque density gradient centrifugation (Lan, K et al., Isolation of human peripheral blood mononuclear cells (PBMCs), Curr Protoc Microbiol Appendix 4, Appendix 4C, 2007). The cells were seeded in 96-well plates at a density of 3×10⁴ cells per well in an RPMI medium including 10% fetal bovine serum (FBS) (Gibco) and cultured for 24 hours. Then, the PBMCs were treated with phosphate-buffered saline (PBS), Cas9-LMWP (13.5 pmol), Cas9 RNPs/Lipofectamine 2000 complex (13.5 pmol), and triplexed Cas9 RNPs (13.5 pmol). In addition, 5 μM CpG oligodeoxynucleotide (ODN) and 50 ng/ml lipopolysaccharide (LPS) were used to induce IFN-α and TNF-α, respectively, as positive controls. After 5 hours of treatment, supernatants of the cells were collected and releases of IFN-α and TNF-α were detected using human IFN-α and TNF-α ELISA kits (Abcam), respectively according to the manufacturer's instructions.

Cell Culture and Transfection

A549 cell lines were obtained from the American Type Culture Collection (ATCC) and maintained in an RPMI 1640 medium supplemented with 10% FBS and 1% penicillin/streptomycin (Gibco) in the presence of 5% CO₂ at 37° C. The cells were plated on 6-well plates at a density of 3×10⁵ cells per well and transfected at the indicated concentrations with Cas9 RNPs (no LMWP)/Lipofectamine 2000 (Invitrogen) or triplexed Cas9 RNP. After 3 hours of incubation, the medium was replaced with an RPMI medium supplemented with 10% FBS. The cells were incubated at 37° C. for 48 hours with 5% CO₂.

Cellular Uptake of Cas9 RNPs

Cellular internalization of the triplexed Cas9 RNPs was evaluated using a confocal microscope. A549 cells were seeded in a μ-Slide 8 well ibitTreat (ibidi) at a density of 1×10⁴ cells/well and cultured for 24 hours. The cells were treated with triplexed Cas9 RNPs at 37° C. with 5% CO₂ in a serum-free medium for 2 hours. Then, the cells were fixed in 4% paraformaldehyde for 20 minutes and permeabilized for 1 hour in 0.2% Triton X-100/2% BSA/PBS. The cells were incubated with a His-tag primary antibody (1:400 dilution) (D3I10, Cell Signaling) for 1 hour and then incubated with an Alexa Fluor 488 secondary antibody (1:1000 dilution) for 1 hour to visualize the triplexed Cas9 RNP. Nuclei were stained with DAPI (300 nM) and analyzed using a confocal microscope (Zeiss LSM 700, Carl Zeiss).

Western Blotting

The treated cells or tumor tissues were lysed using an RIPA lysis buffer (Sigma) unsupplemented or supplemented with a phosphatase inhibitor+a protease inhibitor (Thermo Scientific). A total of 50 μg of protein was analyzed by immunoblotting via incubation at 4° C. overnight with Sprimary antibody specific to KRAS (Santa Cruz, 1:200 dilution), p-ERK (cell signal, 1:1000 dilution), ERK (Cell Signaling, 1:1000 dilution), and β-Actin (Abcam, 1:10000 dilution) and then incubation with secondary antibodies conjugated with horseradish peroxidase for 2 hours at room temperature. The proteins were visualized by chemicalluminescence using a Western ECL substrate (Bio-Rad) and luminescence images were analyzed by a LAS-3000 (Fujifilm). Band intensities were quantified using ImageJ software.

Apoptosis Assay

After treatment with the triplexed Cas9 RNPs (72 pmol), the cells were incubated with Alexa-Fluor 488-conjugated annexin V and PI using an Alexa Fluor 488 annexin V/Dead Cell Apoptosis Kit (Invitrogen) according to the manufacturer's protocol to evaluate induction of apoptosis in cells. Then, stained cells were immediately obtained using a Guava easyCyte™ Flow cytometer (Merck Millipore) and analyzed using a FlowJo version 10.2 (TreeStar). To examine distribution of apoptotic cells using a microscope, cells stained with annexin V and PI were visualized using a confocal microscope (Zeiss LSM 700, Carl Zeiss).

Cell Migration Assay

Cell migration was detected using an Oris™ cell migration analysis kit (Platypus Technologies). The cells were seeded in 96-well plates at a concentration of 5×10⁴ cells per well and maintained overnight. The cells were treated with the triplexed Cas9 RNPs (13.5 pmol) in a serum-free medium for 3 hours and then the culture medium was replaced. The cells were incubated overnight at 37° C. with 5% CO₂, and the stoppers were carefully removed to allow the cells to move into a detection zone. After one more day of incubation, the migrated cells were stained with calcein acetoxymethyl ester (Calcein-AM, BioVision) for 20 minutes and visualized under a fluorescence microscope (Olympus).

Cell Viability Assay for Single or Combination Therapy

The cells were seeded in 96-well plates at a concentration of 3×10³ cells per well and incubated in a growth medium. After overnight incubation, the cells were transfected with triplexed Cas9 RNP (13.5 pmol) and incubated for 24 hours. Then, the cells were further incubated with 5.14 μM AZD6244 (InvivoGen) for 48 hours. Cell viability was evaluated by adding 10 μL of a Cell Counting kit 8 (CCK-8) (Dojindo) at 37° C. for 2 hours, and measured using a micro plate reader (Spectra MAX 340, molecular device) at 450 nm. IC₅₀ value was calculated using Prism 5.02 (GraphPad software). Combination therapy was performed at a fixed IC₃₀ ratio and a combination index (CI) was determined using CalcuSyn software.

Production of A549 Tumor Xenograft Animal Model

All animal care and in vivo experimental procedures were performed in accordance with the regulations of the Institutional Animal Care and Use Committee of the Korea Institute of Science and Technology (KIST). For generation of A549 xenografts, 1×10⁷ cells, mixed in a 50% Matrigel solution (BD Biosciences), were subcutaneously injected into the left flanks of female nude BALB/c mice. When the tumor reached 0.1 to 0.2 cm³, all experiments were performed. For comparison between gene editing in vivo and cationic lipid transfection-based delivery, 0.4 mg/kg of the Cas9 RNPs/Lipofectamine 2000 complex or the triplexed Cas9 RNPs was injected into the tumors twice. Then, the mice were sacrificed at 9 days after the initial treatment and further experiments were performed. To identify in vivo anticancer effects, the triplexed Cas9 RNP was injected into the tumors at a dose of 0.4 mg/kg with or without oral administration of 25 mg/kg of AZD6244. All treatments were performed three times at an interval of three days. Individual tumor volumes were monitored at every three days for four weeks and determined using the equation V=(A×B²)/2, where A is the largest diameter and B is the smallest diameter B. The mice were sacrificed and dissected for further investigation at 28^{th} days after the treatment. To evaluate antitumor effects, tumor growth inhibition rate (% TGI) was determined using the following equation: % TGI=[1−(treated RTV/control RTV)]×100, where RTV is a relative tumor volume.

H&E, TUNEL, and Immunofluorescence Staining

For histological observations, tumors were fixed in 4% paraformaldehyde overnight, embedded in paraffin, and sliced into 6 μm sections using a microtome (Leica). The sections were deparaffinized using xylene and rehydrated using graded ethanol washes. The tumor sections were stained using hematoxylin and eosin (H&E) and observed using an optical microscope. The TUNEL staining was performed using an in situ cell death detection kit (Roche) to evaluate apoptosis in vivo. Particularly, the tumors collected from the mice were embedded in an Optimal Cutting Temperature (OCT) compound (Leica) and frozen at −20° C. The tumor tissue sections were fixed in 4% paraformaldehyde and permeabilized with 0.1% Triton X-100 and 0.1% sodium citrate. Then, the tumor sections were incubated with a TUNEL reaction mixture in a humidified atmosphere at 37° C. for 1 hour in a dark room. After washing three times with PBS, slides were mounted with a DAPI mounting medium (Vector Laboratories) and observed using a confocal microscope (Zeiss LSM 700). For immunostaining in vivo, frozen tissue sections embedded in OCT were stained. The sections were incubated in 0.5% Triton X-100 in PBS and blocked for 2 hours in PBS including 0.1% Triton X-100 and 1% BSA. The sections were incubated at 4° C. overnight with primary antibodies against to KRAS (Santa Cruz, 1:50 dilution), p-AKT (Cell Signaling, 1:200 dilution), and p-ERK (Cell Signaling, 1:250 dilution). After being washed with 0.2% Triton X-100 in PBS, the slides were incubated with secondary antibodies conjugated with Alexa Fluor 488 or Alexa Fluor 594. The slides were mounted on a DAPI mounting medium and observed using a confocal microscope.

### Example 1. Preparation of Triplexed Cas9 RNPs

1.1. Preparation of Cas9-LMWP Expression Vector

To clone LMWP into a Cas9 expression vector, two DNA fragments were amplified from a backbone of a pET28-Cas9-NLS-6×His vector (Addgene #62933) by using primer pairs LMWP-F1/R1 and LMWP-F2/R2. Next, each of the two DNA fragments was partially overlapped with an LMWP-encoding sequence. Then, a second PCR was performed to amplify a long DNA fragment including the NLS and the LMWP using a primer pair LMWP-F1/R2. The DNA fragment including SacI and AvrII ends (compatible ends) was inserted into the backbone plasmid. All enzymes were obtained from New England Biolabs (NEB).

1.2. Purification of Cas9 and Cas9-LMWP Fusion Protein

E. coli BL21 cells were transformed with pET-Cas9-NLS-6×His and pET-Cas9-NLS-LMWP-6×His plasmids and incubated overnight at 37° C. on a Luria-Bertani (LB) agar plate including 100 μg/ml of ampicillin. To induce expression of Cas9 and Cas9-LMWP protein, the transfected BL21 cells were incubated overnight at 18° C. in 400 ml of an LB-ampicillin medium supplemented with 0.2 mM isopropyl-β-D-thiogalactopyranoside (IPTG). The cells were collected by ultracentrifugation and lysed by sonication in a lysis buffer (50 mM Tris (PH 8.0), 100 mM NaCl, 5% glycerol, 5 mM imidazole, and 1 mM phenylmethylsulfonyl fluoride (PMSF)). After ultracentrifugation at 4° C. at 18,000 rpm for 40 minutes, soluble lysates were incubated with an Ni-NTA resin (Thermo Fisher Scientific) at 4° C. for 2 hours and purified using a poly-prep chromatography column (Bio-Rad). The column-binding protein was eluted using a lysis buffer (50 mM Tris (pH 8.0), 50 mM NaCl, 5% glycerol, 300 mM imidazole, and 1 mM PMSF)), and impurities were removed by using an ultrafiltration spin column (Millipore). Purity of the Cas9 and Cas9-LMWP protein was determined with an SDS-PAGE gel.

1.3. Self-Assembly of Triplexed Cas9 RNPs

Two specific crRNAs targeting KRAS and tracrRNA were synthesized by Integrated DNA Technologies (IDT). For hybridization of the dual RNAs, crRNA and tracrRNA in equal molar amounts were incubated in an IDT Duplex buffer (100 mM potassium acetate, 30 mM HEPES, pH 7.5) for 5 minutes at 95° C. and slowly cooled at 20° C. Cas9-LMWP protein and dual RNA duplex (crRNA:tracrRNA) were incubated in serum-free RPMI or PBS buffer at 37° C. for 30 minutes to prepare triplexed Cas9 RNP.

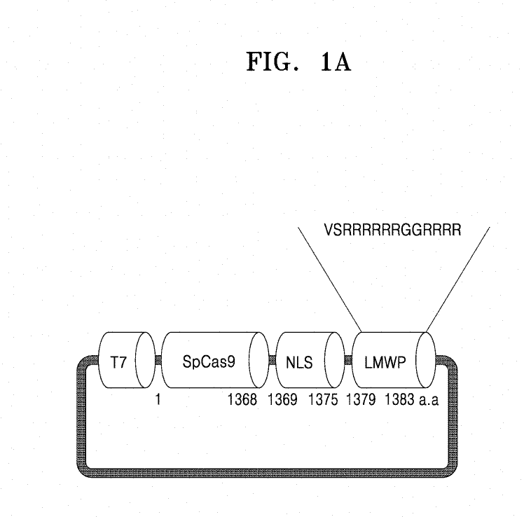

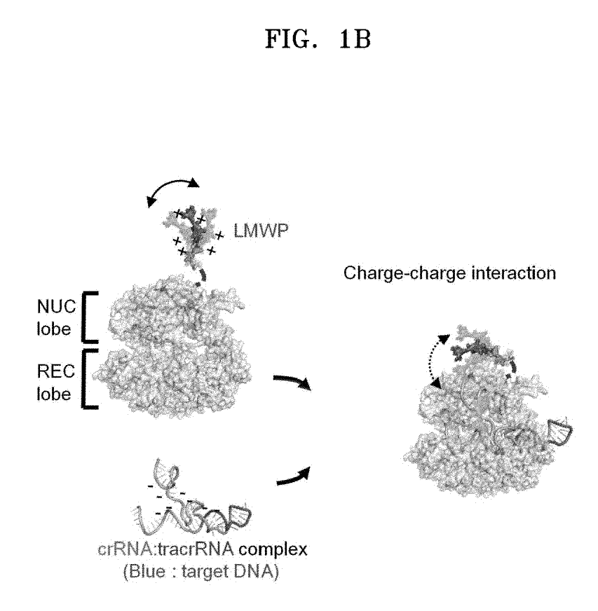

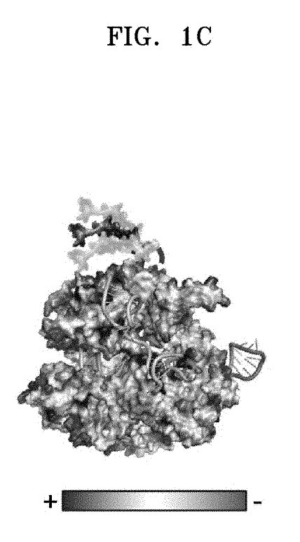

FIG. 1A is a schematic diagram illustrating a vector to prepare triplexed Cas 9 RNPs according to an embodiment. As illustrated in FIG. 1A, NLS and LMWP are located in the C-terminal.

FIG. 1B is a diagram illustrating a structure of triplexed Cas 9 RNPs according to an embodiment. LMWP is shown in red and fused to the C-terminal of Cas9 shown as a grey molecular surface.

FIG. 1C is a diagram illustrating electrostatic surface potential of Cas9 with dual RNAs and target DNA.

As shown in FIGS. 1A to 1C, the NLS mediates nuclear localization thereof for functional editing and the LMWP having high arginine content enables self-assembly of triplexed (Cas9-LMWP/crRNA/tracrRNA) (hereinafter, referred to as “triplexed Cas9 RNPs”) via electrostatically driven interactions and cellular internalization. In addition, a Cas9 expressing both NLS and LMWP may function as a complexing agent and a delivery carrier.

### Experimental Example 1. Characterization of Triplexed Cas9 RNP

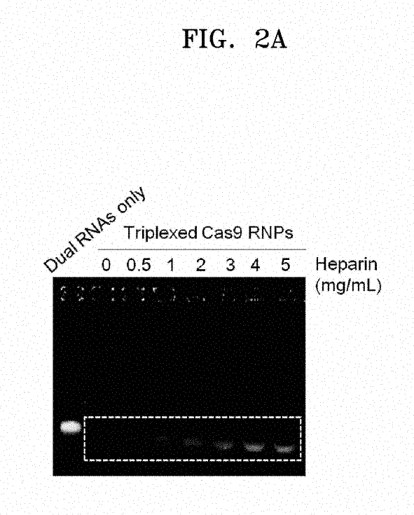

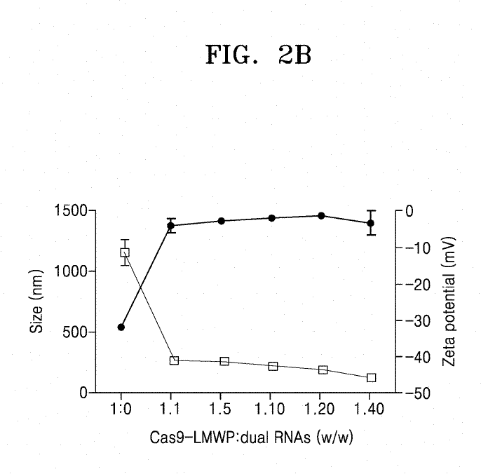

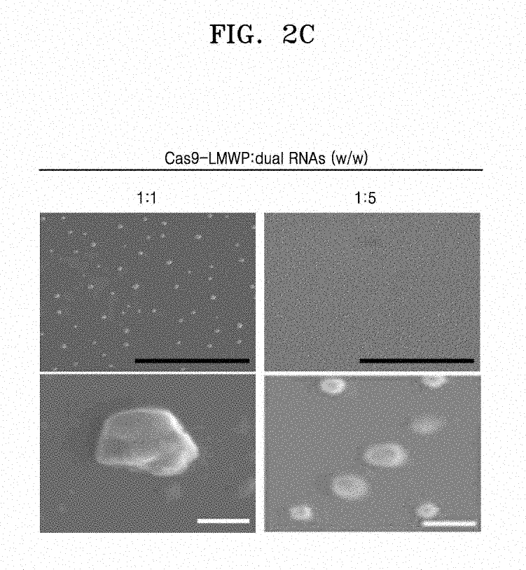

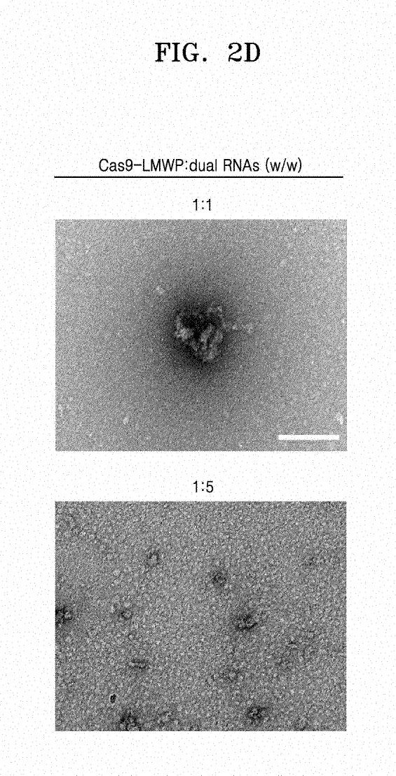

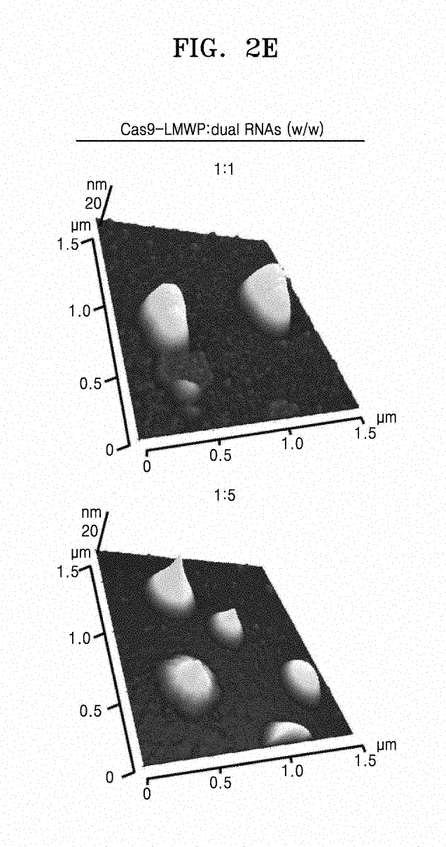

A hydrodynamic size and zeta potential of the triplexed Cas9 RNP were measured on a Zetasizer Nano ZS (Malvern Instruments) and were analyzed using Zetasizer software 7.03. For gel mobility shift assay, different concentrations of heparin, ranging from 0 to 5 mg/ml, were added to 10 μL of a triplexed Cas9 RNP-containing solution and incubated at 37° C. for 15 minutes. The resulting band shifts were visualized by 1% agarose gel electrophoresis. Morphologies and sizes of the complex were analyzed using transmission electron microscopy (TEM) (CM30 electron microscope, Philips). Atomic Force Microscopy was performed using an XE-100 (Park Systems). Scanning electron microscopic (SEM) images were obtained from an FEI Teneo Volume scope using a voltage of 10 kV. All experiments were quantitatively analyzed via ImageJ software (National Institutes of Health) and the results are shown in FIGS. 2A to 2E.

FIG. 2A is a diagram illustrating electrostatically induced complex formation of Cas9-LMWP fusion protein and dual RNA identified by a gel mobility shift assay.

FIG. 2B is a graph illustrating zeta potentials (red lines) and size distributions (blue lines) of resulting complexes respectively measured with a Zetasizer and DLS.

FIG. 2C shows SEM images illustrating the morphologies, size distributions, and size homogeneity of triplexed Cas9 RNPs at two given ratios (1:1 or 1:5).

FIG. 2D shows TEM images illustrating the morphologies, size distributions, and size homogeneity of triplexed Cas9 RNPs at two given ratios (1:1 or 1:5).

FIG. 2E shows AFM images illustrating the morphologies, size distributions, and size homogeneity of triplexed Cas9 RNPs at two given ratios (1:1 or 1:5).

As illustrated in FIG. 2A, it may be confirmed that a high dose of heparin abolished the interaction of Cas9-LMWP with the dual RNAs. In addition, as illustrated in FIG. 2B, after self-assembly of the triplexed Cas9 RNPs, a surface net charge increased from −30 mV to −4 mV.

Based on the above results, it may be confirmed that highly enriched cationic LMWP on Cas9-LMWP self-assembled with anionic dual RNAs regardless of addition of any cationic polymers.

In addition, as illustrated in FIGS. 2C to 2E, while the complex shows approximately 159 nm at a ratio of 1:1, the complex was approximately 89 nm at a ratio of 1:5, based on the SEM analysis. The self-assembled complex at the ratio of 1:5 was more densely formed than at the ratio of 1:1. Thus, it may be confirmed that the size of complex with the Cas9-LMWP and the dual RNA may be manipulated by using precisely defined ratios.

### Experimental Example 2. Analysis of Immunogenicity of Triplexed Cas9 RNP

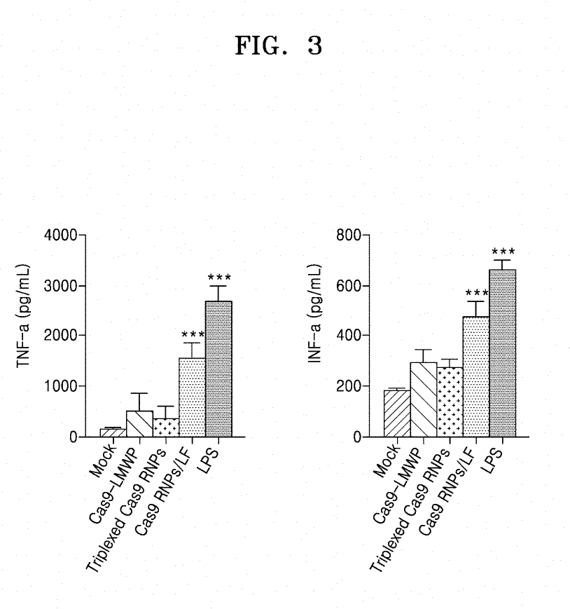

To determine immunogenicity induced by the triplexed Cas9 RNP, releases of TNF-α and IFN-α were detected according to Reference Example as described above and the results are shown in FIG. 3.

FIG. 3 is a diagram illustrating immunogenicity of triplexed Cas9 RNP according to an embodiment. *P<0.05, ** P<0.01, *** P<0.001 by a one-way ANOVA followed with Tukey's multiple comparison test.

As shown in FIG. 3, it was confirmed that Cas9-LMWP or triplexed Cas9 RNP caused less immunogenicity and the Cas9 RNPs complexed with LF2000 increased induction of large amounts of cytokines.

These results indicate that the Cas9-LMWP is a safe carrier since the amount of TNF-α or IFN-α is considerably reduced by treatments with the Cas9-LMWP alone or triplexed Cas9 RNP when compared with treatments with a conventional transfection reagent, LF2000.

### Experimental Example 3. Analysis of In Vitro Anticancer Effect

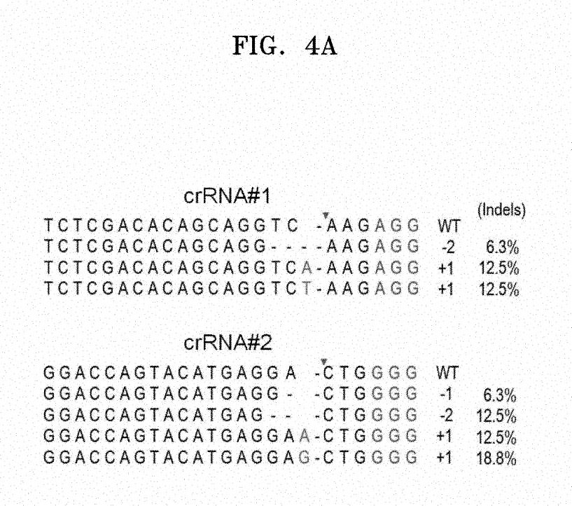

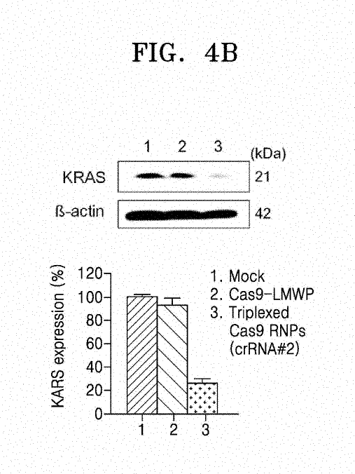

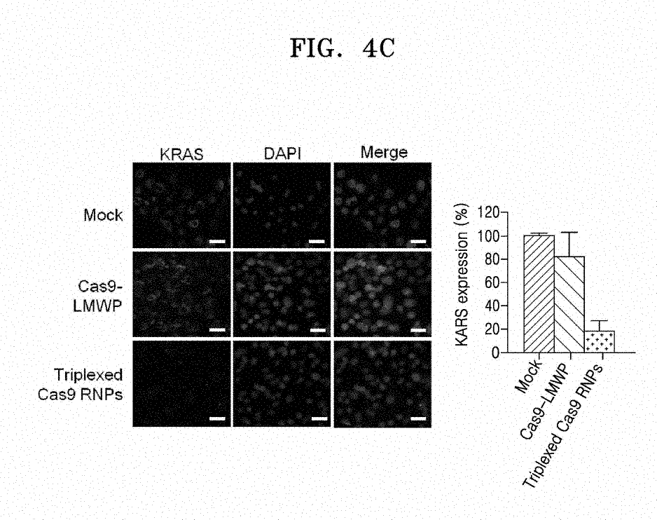

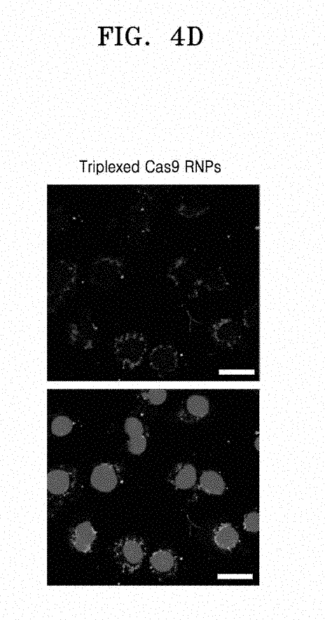

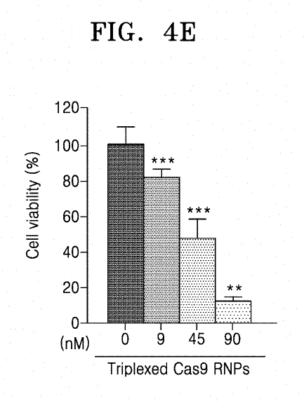

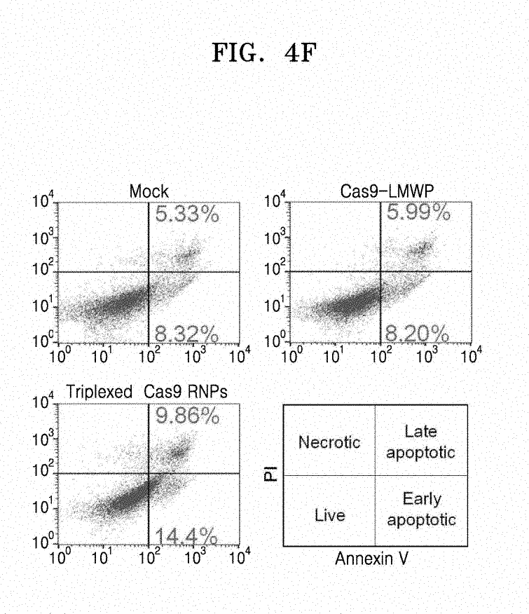

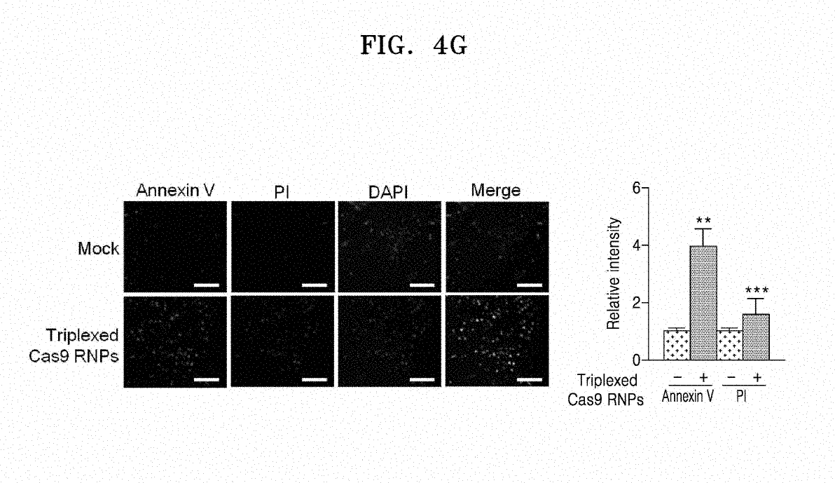

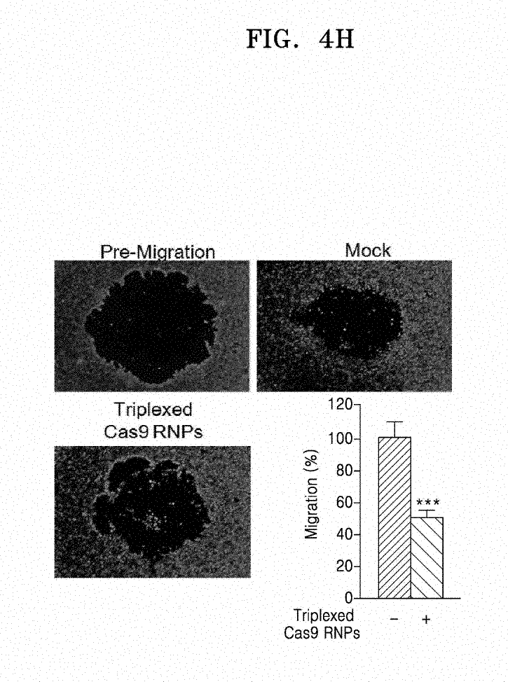

To identify whether the triplexed Cas9 RNPs are functional, insertion/deletion (indel) mutations induced by the triplexed Cas9 RNPs were evaluated by using two different crRNAs in human NSCLC A549 cells (Refer to 4A). Particularly, as described above in Reference Example, anticancer effects on Cas9 RNP-mediated KRAS disruption among lung cancer cells were analyzed, and the results are shown in FIGS. 4B to 4H.

FIG. 4A is a diagram illustrating frequencies of indel mutations in A549 cells treated with Cas9-LMWP and crRNA #1 or crRNA #2.

FIG. 4B is a diagram illustrating effects of triplexed Cas9 RNPs (72 pmol) on suppressing expression of KRAS identified by immunoblotting.

FIG. 4C illustrates confocal microscopic images indicating suppression of KRAS expression after direct delivery of triplexed Cas9 RNPs (72 pmol) (left panel) and a graph obtained by quantifying the results (right panel).

FIG. 4D illustrates cellular internalization of triplexed Cas9 RNPs (72 pmol). Green and red colors indicate locations of Cas9-LMWP from triplexed Cas9 RNPs and nuclei, respectively.

FIG. 4E is a graph illustrating viability of lung cancer cells according to various doses of triplexed Cas9 RNPs.

FIG. 4F illustrates FACS analysis results indicating apoptosis induced by triplexed Cas9 RNPs (72 pmol).

FIG. 4G illustrates confocal microscopic images indicating apoptosis induced by triplexed Cas9 RNPs (72 pmol) (left panel) and a graph obtained by quantifying the results (right panel).

FIG. 4H is a diagram illustrating that triplexed Cas9 RNPs (13.5 pmol) significantly suppresses cell migration. *P<0.05, ** P<0.01, *** P<0.001 by a one-way ANOVA followed with Tukey's multiple comparison test.

As illustrated in FIG. 4A, the frequencies of the indel mutations were detected at loci in KRAS in the A549 cells treated with Cas9-LMWP and crRNA#1 or crRNA#2. In addition, as illustrated in FIGS. 4B and 4C, in agreement with the results from the indel frequencies, it was confirmed that the A549 cells exhibited significant disruption of KRAS protein after 48 hours from treatment with crRNA#2. In addition, as illustrated in FIG. 4D, confocal microscopic images showed a high accumulation of the triplexed Cas9 RNP in the A549 cells at 2 hours after the treatment. Large spots indicating Cas9 RNPs and labeled with FITC were observed in the cytoplasm and nucleus. Also, as shown in FIG. 4E, cell viability in the A549 cells treated with the triplexed Cas9 RNP, ranging from 4.5 pmol to 50 pmol was tested, and it was confirmed that cell viability gradually decreased in a dose-dependent manner. Also, as illustrated in FIGS. 4F and 4G, it was confirmed that the triplexed Cas9 RNPs induced apoptosis in A549 cells. In addition, as illustrated in FIG. 4H, it was confirmed that Cas9 RNP-based KRAS depletion suppressed cell migration by 54%.

According to the above-described results, it may be confirmed that the triplexed Cas9 RNPs efficiently delivers Cas9 nuclease and dual RNAs into A549 cells without the aid of any transfection reagents, thereby inducing apoptosis and suppressing cell migration.

### Experimental Example 4. Analysis of Triplexed Cas9 RNPs-Mediated Delivery Ability for Gene Editing In Vitro and In Vivo

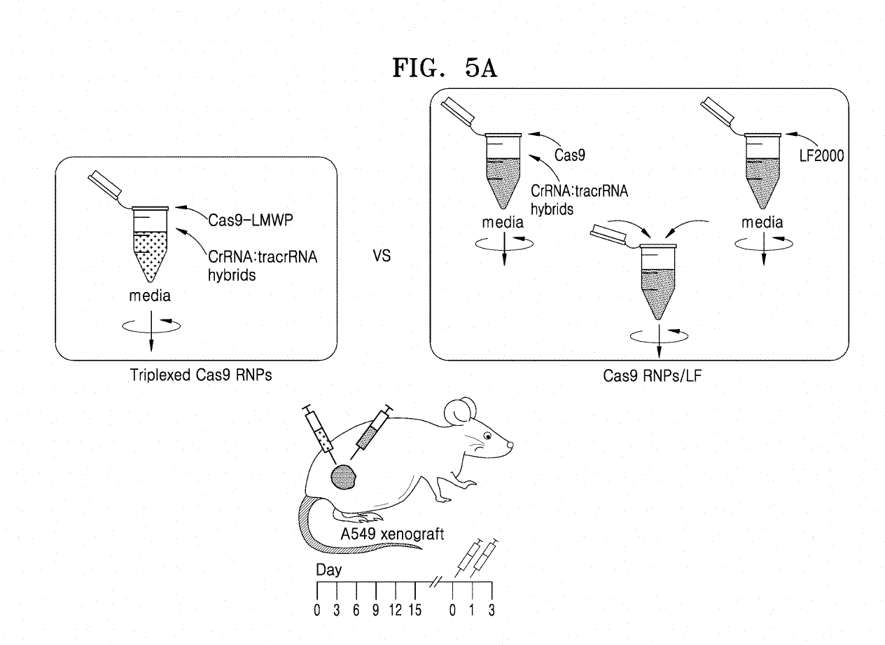

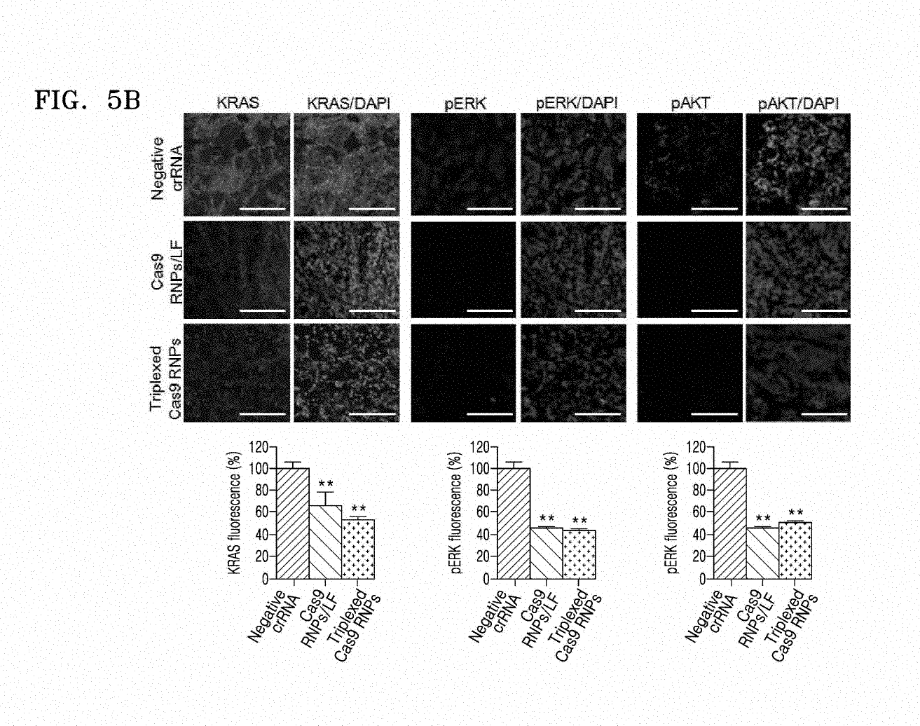

To further validate efficient gene editing in vivo, the LF2000/Cas9 RNPs (without LMWP) and triplexed Cas9 RNPs were administered into A549 xenograft models as described above in Reference Example, and the results are shown in FIGS. 5A and 5B.

FIG. 5A is a diagram illustrating a scheme of administering triplexed Cas9 RNPs and LF2000/Cas9 RNPs into A549 xenograft tumor models for comparison of capabilities of delivering substances for in vivo gene editing.

FIG. 5B illustrates confocal microscopic images showing results of administration of triplexed Cas9 RNPs and LF2000/Cas9 RNPs (upper panel) and graphs obtained by immunofluorescence staining analysis (lower panel). Bars indicate 50 μm, and inhibition of KRAS, p-ERK, and p-AKT was quantified using imageJ program (N=3). *P<0.05, ** P<0.01, *** P<0.001 by a one-way ANOVA followed with Tukey's multiple comparison test.

As illustrated in FIG. 5B, although both treatments showed similar efficiencies of gene editing against KRAS, it was confirmed that groups treated with the triplexed Cas 9 RNPs had significant inhibition in p-AKT and p-ERK, which are downstream molecules of KRAS, in comparison with groups treated with the LF2000/Cas9 RNP. Thus, it was confirmed that the triplexed Cas 9 RNPs increased gene editing in vivo efficiency.

### Experimental Example 5. Analysis of Synergic Effects by Co-Treatment with Anticancer Agent

To establish, effective anti-KRAS cancer therapy, the triplexed Cas9 RNPs were co-treated with an anticancer agent.

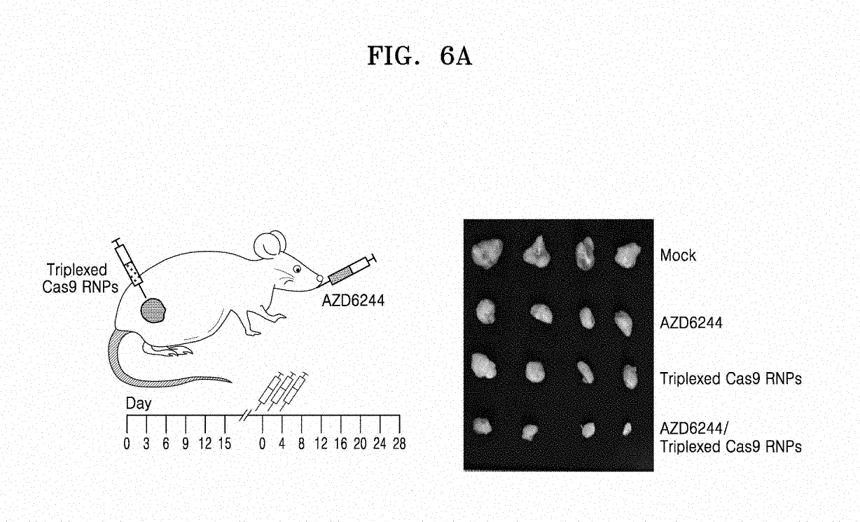

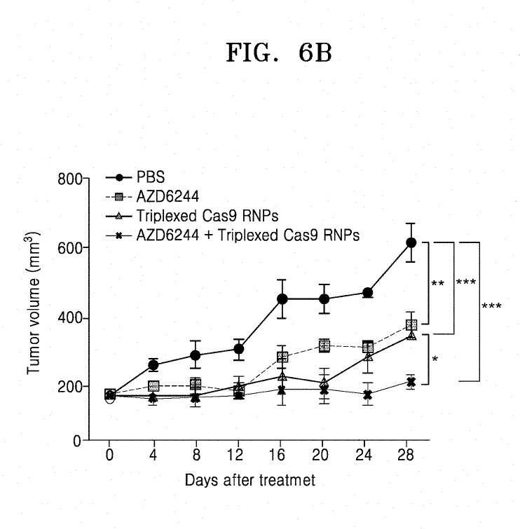

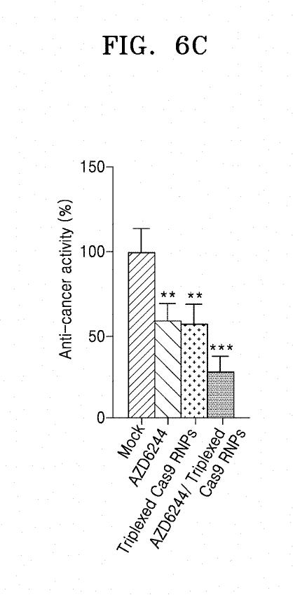

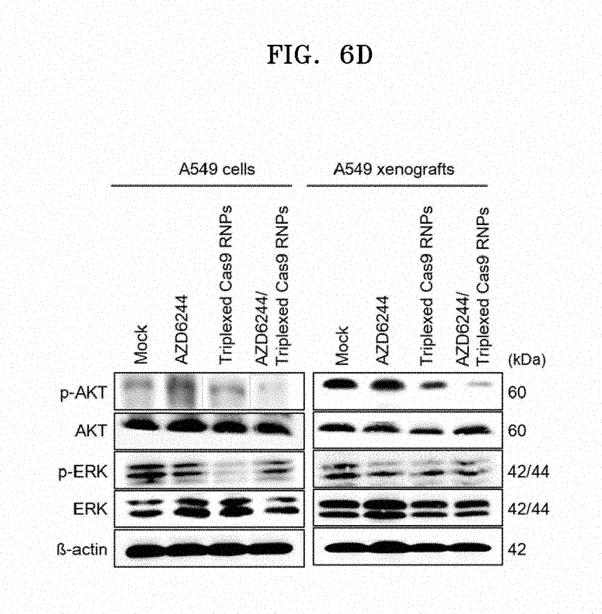

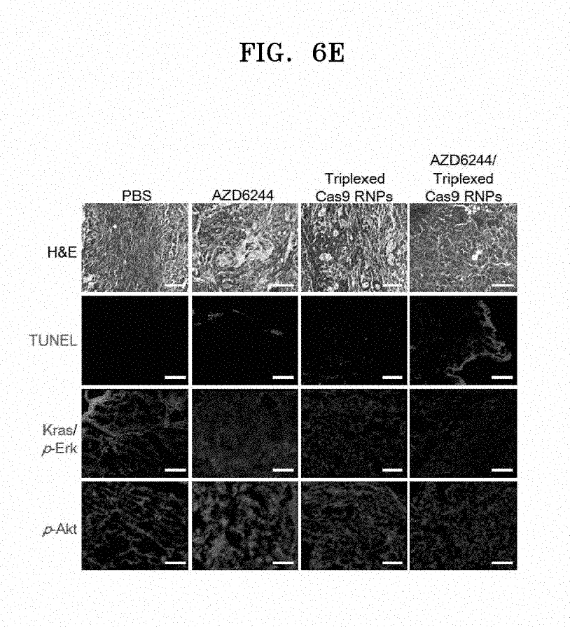

Particularly, the experiment was performed using AZD6244 that is an MEK inhibitor blocking ERK activation as described above in Reference Example, and the results are shown in FIGS. 6A to 6E.

FIG. 6A illustrates a scheme of administering triplexed Cas9 RNPs (0.4 mg/kg) and AZD6244 (25 mg/kg) by either single treatment or co-treatment (left panel) and photographs of tumors isolated from mice at 28 days after treatment.

FIG. 6B is a graph illustrating sizes of tumors after single treatment or co-treatment of the triplexed Cas9 RNPs (0.4 mg/kg) and AZD6244 (25 mg/kg). *P<0.05, ** P<0.01, *** P<0.001 by a one-way ANOVA followed with Tukey's multiple comparison test.

FIG. 6C is a graph illustrating tumor growth inhibition (% TGI) for evaluation of antitumor efficiency. *P<0.05, ** P<0.01, *** P<0.001 by a one-way ANOVA followed with Tukey's multiple comparison test.

FIG. 6D illustrates immunoblot analysis of EGFR downstream signaling pathways in A549 cells and A549 xenografts.

FIG. 6E illustrates tumor cells stained by H&E staining, TUNEL staining, and immunolabelling of p-ERK, KRAS, and p-AKT after single treatment or co-treatment of the triplexed Cas9 RNPs (0.4 mg/kg) and AZD6244 (25 mg/kg). Bars indicate 50 μm.

As illustrated in FIGS. 6A to 6E, it was confirmed that extensive synergistic effects (CI<1, 0.34) were obtained in A549 cells by co-treatment of AZD6244 and triplexed Cas9 RNPs. Since the MEK1/2-ERK pathway is one of the downstream pathways, the strong synergistic interactions of the triplexed Cas9 RNPs-mediated KRAS depletion and the MEK inhibitor may provide a rationale for the use of combination therapy in KRAS or KRAS/PTEN mutated cancers. In addition, as illustrated in FIGS. 6B and 6C, a significant decrease in tumor growth was confirmed in the A549 xenograft model co-treated with AZD6244 and triplexed Cas9 RNPs. As a result of measuring the tumor growth inhibition as illustrated in FIG. 6D, it was confirmed that co-treatment led to a TGI of 73%. Also, as illustrated in FIG. 6E, the immunoblotting assay was performed in in vitro and in vivo samples to evaluate signal transfer mechanisms that underlie the anti-KRAS therapy. As a result, the administration of AZD6244 inhibited only activation of ERK, but not AKT. In contrast, the co-treatment affected phosphorylation of both AKT and ERK. Since the depletion of KRAS affected the AKT/STAT3 pathway as well as the MAPK signaling pathway, the combination therapy exhibited synergistic in vivo and in vitro anticancer effects by inhibiting activation of AKT and ERK.

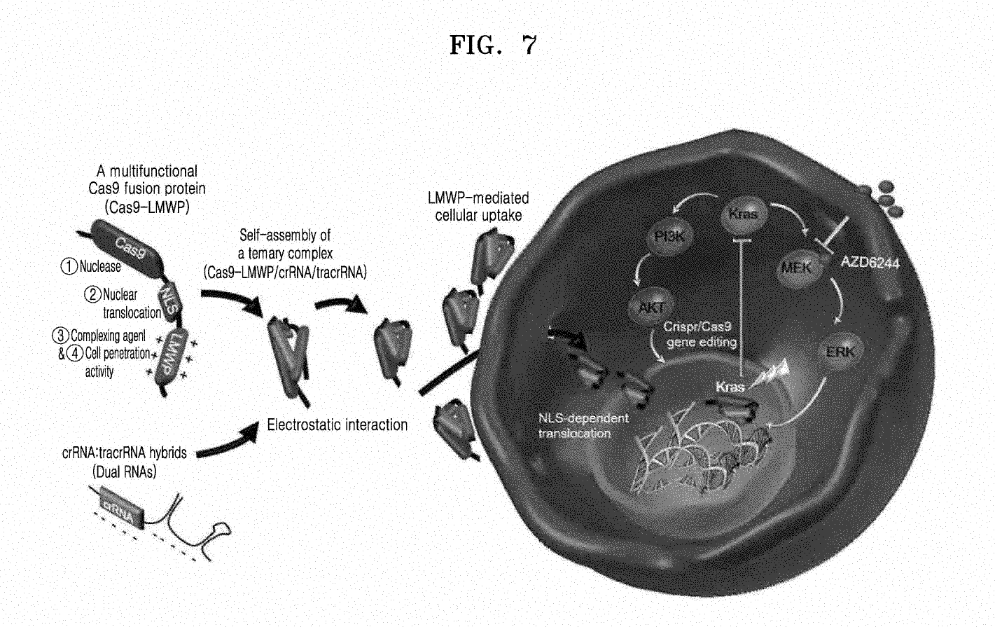

FIG. 7 is a diagram illustrating triplexed Cas9 RNPs-mediated gene editing for cancer therapeutics according to an embodiment.

As illustrated in FIG. 7, the triplexed Cas9 RNPs according to an embodiment may be used as a delivery medium for gene editing by efficiently delivering dual RNAs (crRNA:tracrRNA hybrid) into cells. The LMWP may function as a cell penetrating peptide and the triplexed Cas9 RNPs may deliver the Cas9 nuclease and dual RNA into the nucleus owing to the existence of the NLS. That is, the Cas9-LMWP fusion protein is an RNA-programmed nuclease having cell penetrating activity and nuclear translocation properties as well as a complexing agent. In addition, synergistic anticancer effects may be obtained by combining therapeutic gene editing against KRAS and the MEK inhibitor that inhibits multiple signaling transductions including the PI3K/AKT.

The fusion protein according to an embodiment may efficiently be used as an anticancer agent due to formation of a complex with a guide RNA, higher in vivo or in vitro delivery activity of guide RNA than e methods without any other cationic polymers or lipid carriers, and synergistic effects by co-treatment with any other anticancer agent as well as anticancer activity by single treatment.

It should be understood that embodiments described herein should be considered in a descriptive sense only and not for purposes of limitation. Descriptions of features or aspects within each embodiment should typically be considered as available for other similar features or aspects in other embodiments.

While one or more embodiments have been described with reference to the figures, it will be understood by those of ordinary skill in the art that various changes in form and details may be made therein without departing from the spirit and scope of the disclosure as defined by the following claims.

What is claimed is:

1. A fusion protein comprising:
a CRISPR-associated protein (Cas protein);
a nuclear localization sequence (NLS); and
a cationic cell penetrating peptide.

2. The fusion protein of claim 1, wherein the Cas protein is a Cas9 protein.

3. The fusion protein of claim 1, wherein, the cationic cell penetrating peptide is a low molecular weight protamine (LMWP).

4. The fusion protein of claim 1, wherein the NLS binds to a C-terminal of the Cas protein, and the cationic cell penetrating peptide binds to a C-terminal of the NLS.

5. A host cell transformed by an expression vector including a nucleic acid molecule encoding the fusion protein according to claim 1.

6. The host cell of claim 5, wherein the cell is selected from group consisting of a cell of yeast, fungus, protozoa, plant, insect, amphibian and mammalian.

7. A complex comprising:
a fusion protein comprising a CRISPR-associated protein (Cas protein) and a cationic cell penetrating peptide; and
a guide RNA.

8. The complex of claim 7, wherein the guide RNA is a dual RNA comprising a CRISPR RNA (crRNA) and a transactivating crRNA (tracrRNA), or a single-stranded guide RNA (sgRNA) comprising portions of the crRNA and the tracrRNA and hybridizing with a target DNA.

9. The complex of claim 8, wherein the crRNA binds to the tracrRNA.

10. The complex of claim 7, wherein the complex targets a sequence of KRAS represented by SEQ ID NO: 10.

11. The complex of claim 7, wherein the fusion protein further comprises a nuclear localization sequence (NLS).

12. The complex of claim 7, wherein the complex is self-assembled by the fusion protein to form a complex with a guide RNA.

13. A method of preparing a complex to deliver a guide RNA into a cell including brining the fusion protein according to claim 1 into contact with the guide RNA.

14. A method of treating cancer, the method including administering a pharmaceutical composition comprising the complex according to claim 8 into an individual in need thereof.

15. The method of claim 14, the pharmaceutical composition further comprising a targeted anticancer agent.

16. The method of claim 14, wherein the cancer is selected from lung cancer, non-small-cell lung cancer, pancreatic cancer, gastric cancer, liver cancer, colon cancer, brain cancer, breast cancer, thyroid cancer, bladder cancer, esophageal cancer, or uterine cancer.

17. The method of claim 14, wherein the complex targets a sequence of KRAS represented by SEQ ID NO: 9.
